(19) United States
(12) Patent Application Publication
(10) Pub. No.: US 2007/0116662 A1
(43) Pub. Date: May 24, 2007

(54) Antiperspirant/deodorant compositions

(76) Inventors: James Zielinski, Somerset, NJ (US); Peter Christian Oehler, Houston, TX (US); Kathleen Kim Cooper, South River, NJ (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 11/284,274

(22) Filed: Nov. 21, 2005

(51) Int. Cl. A61K 8/81

(52) U.S. Cl. 424/70.16

(57) ABSTRACT

Disclosed herein is a composition for topical. application to human skin, comprising a biologically active material and a cosmetically suitable carrier, wherein the cosmetically suitable carrier comprises 5-95 wt % of a mixed feed polyolefin based on the total weight of the composition, wherein the mixed feed polyolefin comprises two or more C₆-C₂₀ copolymerized monomers, and wherein at least two of the copolymerized monomers are each present in the mixed feed polyolefin at 10 wt % or more, based on the total weight of the mixed feed polyolefin. A method of making a composition for topical application to human skin is also disclosed.

### FIELD OF THE INVENTION

This invention relates to cosmetic compositions suitable for topical application to the human skin. In particular the cosmetic compositions comprise an antiperspirant or deodorant active material and a mixed feed polyolefin, preferably a poly-α olefin. This invention further relates to cosmetic compositions suitable for topical application to the human skin. In particular, it relates to antiperspirant and deodorant compositions for topical application to any appropriate area of the human body, such as the axilla.

### BACKGROUND OF THE INVENTION

Antiperspirant and deodorant compositions have long been known for topical application to the body, in particular the axilla, for the control of perspiration and/or odor. Such products may take the form of aerosol compositions, a lotion, a stick, a cream, a pump spray formulation, and the like. Antiperspirant compositions contain an antiperspirant active salt, which is typically aluminum or aluminum/zirconium salt in a topically acceptable carrier, which acts to prevent the pores of the skin from secreting sweat. Deodorant compositions typically comprise a deodorant agent in a cosmetically acceptable carrier. The deodorant agent may be an inorganic salt which has a deodorant effect, such as a zinc or an aluminum salt, or may be an organic material having deodorant efficacy. Examples of organic deodorant agents include short chain monohydric alcohols such as ethanol, polyhydric alcohols such as propylene glycol, and/or specific compounds such as triclosan.

Volatile silicones were introduced as a substitute for ethanol in antiperspirant and deodorant formulations more than 20 years ago. Examples include cyclomethicone and other silicones, which continue to be a major ingredients or component in the cosmetically acceptable carriers used in antiperspirant and deodorant formulations. Although ethanol dries off of the skin quicker than cyclomethicones, ethanol also dissolves lipids on skin that results in an undesirable tight skin-feel. The elegant feel that silicones impart to the product is so far unsurpassed. In addition, silicones serve as effective carriers for aluminum zirconium complexes.

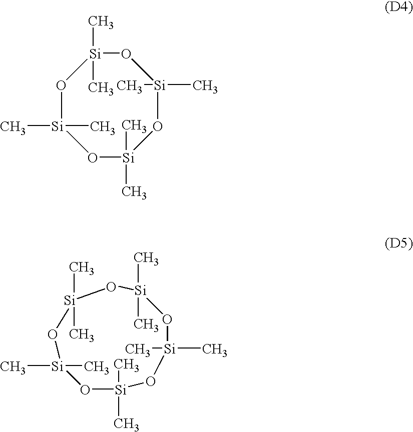

The volatile silicone oils come primarily in two forms, cyclomethicone tetramer (D4) and cyclomethicone pentamer (D5). The use of D4 is limited due to possible toxicology issues directed to possible endocrine disrupter functionality. D5 has also come under scrutiny and more manufactures are looking for a safe, cost effective, and plentiful replacement for D5. Accordingly, a need exists to provide a cosmetically acceptable carrier, which does not comprise cyclomethicone as its major component, if at all.

Particular poly alpha olefins (PAO's) are one possible component that may be used in large proportion in a cosmetically acceptable carrier for deodorant, antiperspirants, and the like. In particular, polydecene provides much of the same carrier properties as other components, without the possible toxicological issues. For example, EP 0 804 921 assigned to Unilever PLC, UK discloses an antiperspirant or deodorant composition comprising an active material and a cosmetically suitable vehicle, which comprises 20-90 weight percent of the total composition, wherein the cosmetically suitable vehicle is a polyolefin emollient.

U.S. Pat. No. 4,919,934 assigned to Richardson-Vicks Inc., discloses a cosmetic stick composition comprising 5-90 weight percent of one or more polyolefins R₁C═CR₂, wherein R₁, R₂═C₂₀₋₇₀ alkyl; having an average molecular weight of 300-800 Daltons and a viscosity between 2-10 centistokes (cSt) at 100° C.

US 2002034481 assigned to Unilever Home and Personal Care USA, discloses a carrier for use in an antiperspirant/deodorant, which comprises a poly olefin and an alkyl benzoate.

The factors which are required to proclaim a composition to be a cosmetically acceptable carrier are wildly varied. Such factors are also difficult if not impossible to quantify. Factors which affect a cosmetically acceptable carrier include the feel of the composition on the skin, the apparent greasiness of the composition, the color, the odor, the refractive index, and the like. Accordingly, formulators of such products are not apt to quickly change formulations, which have demonstrated commercial acceptance. However, in terms of PAOs, the art has centered around polydecene compositions, and/or blends which comprise discrete proportions of polydecene. This demand has created a shortage of polydecene, along with an increase in the price of the material. Accordingly, a need exists for an alternative to polydecene based PAOs which may be used in a cosmetically acceptable carrier in place of polydecene, in a manner which is transparent to the consumer.

### SUMMARY OF THE INVENTION

In one aspect of the present invention, a composition for topical application to human skin, comprises a biologically active material and a cosmetically suitable carrier, wherein the cosmetically suitable carrier comprises 5-95 wt % of a mixed feed polyolefin based on the total weight of the composition, wherein the mixed feed polyolefin comprises two or more C₆-C₂₀ copolymerized monomers, and wherein at least two of the copolymerized monomers are each present in the mixed feed polyolefin at 10 wt % or more, based on the total weight of the mixed feed polyolefin.

In another aspect of the present invention, a method of making a composition for topical application to human skin comprising a biologically active material and a cosmetically suitable carrier, comprises the steps of combining a mixed feed polyolefin into a carrier to produce a cosmetically suitable carrier, wherein the cosmetically suitable carrier comprises 5-95 wt % of the mixed feed polyolefin based on the total weight of the composition, wherein the mixed feed polyolefin comprises two or more C₆-C₂₀ copolymerized monomers, and wherein at least two of the copolymerized monomers are each present in the mixed feed polyolefin at 10 wt % or more, based on the total weight of the mixed feed polyolefin.

A more complete appreciation of the claimed invention and many of the attendant advantages thereof will be readily obtained as the invention becomes better understood by reference to the following detailed description when considered in connection with the accompanying formulas, tables and embodiments. Further, as is made apparent from the general description, preferred embodiments, and illustrated examples, while forms of the invention have been illustrated and described, various modifications can be made without departing from the spirit and scope of the invention. Accordingly, it is not intended that the invention be limited to the specific embodiments described herein.

### DETAILED DESCRIPTION

We have surprisingly found that poly olefins, preferably poly-α olefins (PAOs) produced from mixed feed stocks can be used as a polydecene PAO replacement in antiperspirant, deodorant, and other compositions for topical application. Surprisingly, mixed feed polyolefins, preferably mixed feed PAOs may be used as the major part of the cosmetic carrier, and yet result in products which have remarkably good sensory properties. Mixed feed polyolefins, in particular, mixed feed PAOs have been found to be remarkably lacking in greasiness upon application. In addition, where the composition is in the form of an antiperspirant composition, the inclusion of a mixed feed polyolefin, preferably a mixed feed poly-α olefin has the added benefit of masking white deposits which may be caused by the presence of antiperspirant active salts in the composition, thereby improving the appearance of the applied product in use. It has also been found that the presence of mixed feed polyolefin, preferably a mixed feed poly-α olefins does not interfere with the efficacy of the antiperspirant and/or deodorant composition once applied.

In an embodiment this invention relates to an antiperspirant or deodorant composition for topical application to the human skin, comprising an antiperspirant or deodorant active material and a cosmetically suitable vehicle, the cosmetically suitable vehicle comprising 5-95 wt % of a mixed feed polyolefin, preferably a mixed feed poly-α olefin (MFPAO), based on the total weight of the composition. The mixed feed polyolefin of the present invention comprises two or more C₆-C₂₀ copolymerized monomers, wherein at least two of the copolymerized monomers are each present in the mixed feed polyolefin at 10 wt % or more, based on the total weight of the mixed feed polyolefin.

Mixed Feed Polyolefins and Mixed Feed Poly-Alpha-Olefins

As used herein, mixed feed polyolefins and mixed feed poly-alpha-olefins (mixed feed PAOs, and/or MFPAO) are defined as poly-olefins produced from a mixed olefin feed stream comprising two or more olefin monomers, wherein at least one of the two or more olefin monomers comprises at least 10 wt % of the mixed olefin feed stream. The two or more olefin monomers each comprise at least 6 carbon atoms, preferably 6 to 20 carbon atoms, more preferably 6 to 14 carbon atoms, more preferably 8 to 14 carbon atoms, with a feed stream comprising olefin monomers having 8 to 12 carbon atoms being most preferred.

In an embodiment, the mixed olefin feed stream comprises at least two olefin monomers, preferably at least three olefin monomers, more preferably at least four olefin monomers. At least one of the two or more olefin monomers comprises at least 10 wt % of the mixed olefin feed stream. In another embodiment, at least one of the two or more olefin monomers comprises at least 20 wt %, preferably at least 30 wt %, preferably at least 40 wt %, preferably at least 50 wt % of the mixed olefin feed stream. In still another embodiment, two or more of the olefin monomers comprise at least 10 wt % each of the mixed olefin feed stream, preferably two or more of the olefin monomers comprise at least 20 wt % of the mixed olefin feed stream.

The mixed olefin feed stream may comprise monomers having from 6 to 20 carbon atoms. The monomers comprise at least one carbon-carbon double bond. In an embodiment, the mixed feed polyolefins may comprise linear or branched C₆ to C₂₀ alkyl groups. The monomers may comprise an alpha olefin (i.e., 1-alkene), an internal olefin, or a combination thereof. In a preferred embodiment, the mixed feed polyolefins may comprise or may consist essentially of linear alpha olefins, branched alpha olefins, or a combination thereof. For simplicity herein, mixed feed polyolefins and mixed feed poly-alpha-olefins are simply referred to as mixed feed poly-alpha-olefins, abbreviated as mixed feed PAOs, and/or MFPAOs, unless specifically stated otherwise.

Importantly, the mixed feed PAOs of the present invention comprise a mixture of PAOs which have been oligomerized (polymerized) from a mixture of different monomers. For purposes herein, it is to be understood that the mixed feed PAOs of the present invention are not the same or an obvious variant of simple mixtures comprising a blend or mixture of different discrete poly olefins and/or poly-alpha olefins. Accordingly, the inventive mixed feed PAOs of the present invention are not produced via mixing of discrete poly-alpha olefins. For example, the inventive mixed feed PAO produced from a mixed olefin feed stream comprising C₈, C₁₀, and C₁₂ monomers would not be the same as the blend obtained by mixing discrete quantities of C₈, C₁₀, and C₁₂ PAOs, each produced independently from one another, and each produced from a feed stream comprising essentially one monomer. Accordingly, the inventive mixed feed PAOs may comprise polyolefin chains comprising a mixture of the various monomers contained in the mixed feed olefin stream. However, in an embodiment of the present invention, a mixed feed PAO may be combined with one or more conventional PAOs produced via oligomerization from essentially one monomer.

The ability to produce a mixed feed PAO from a mixed feed stream is thus a benefit in terms of the amount of purification, processing required, and availability in regard to the feed stream prior to oligomerization. In addition, mixed feed PAOs have shown other unexpected properties rendering them suitable for use in, and more particularly as replacements for, PAOs useful in cosmetically acceptable carrier formulations.

The mixed feed polyolefins of the present invention are hydrocarbon polymers. Preferably, the mixed feed PAOs are liquid at room temperature (i.e. 21° C.). In an embodiment, the mixed feed PAOs present in the cosmetically acceptable carrier (e.g., the cosmetic vehicle) will be non volatile. By “non volatile” in this context is meant that the mixed feed PAO has a flash point of 140° C. or greater. In another embodiment, the mixed feed PAOs present in the cosmetically acceptable carrier are volatile. By “volatile” in this context is meant that the mixed feed PAO has a flash point of less than 140° C. In still another embodiment, a mixed feed PAO, (or a conventional PAO) which are non-volatile may be combined with a mixed feed PAO (or a conventional PAO) which are volatile, and/or any combination thereof.

It is also highly preferred that at least a portion of the mixed feed PAO has a relatively low viscosity of less than about 40 cSt at 40° C., preferably less than about 30 cSt at 40° C., preferably less than about 20 cSt at 40° C., preferably less than about 10 cSt at 40° C., preferably less than about 9 cSt at 40° C., preferably less than about 8 cSt at 40° C., preferably less than about 7 cSt at 40° C., preferably less than about 6 cSt at 40° C., preferably less than about 5 cSt at 40° C., preferably less than about 4 cSt at 40° C., preferably less than about 3 cSt at 40° C., more preferably less than about 2 cSt at 40° C. In other embodiments, at least a portion of the mixed feed PAO may have a relatively high viscosity of greater than or equal to about 100 cSt at 40° C., preferably greater than or equal to about 1000 cSt at 40° C. Accordingly, the mixed feed PAO may comprise a blend of discrete mixed feed PAOs, so long as each of the PAOs in the blend qualify as mixed feed PAOs. In still another embodiment, a mixed feed PAO, (or a conventional PAO) having a relatively low viscosity may be combined with a mixed feed PAO (or a conventional PAO) having a relatively high viscosity, and/or any combination thereof. The mixed feed PAO of the present invention may also have a refractive index at 25° C. of 1.4 or greater, preferably 1.45 or greater, with 1.46 or greater being more preferred.

Formation of Mixed Feed PAOs

Mixed feed PAOs may be produced according to U.S. Pat. No. 4,950,822, wherein at column 2, lines 63-66, mixed feed PAOs comprising C₈, C₁₀ and C₁₂ are disclosed. In addition, mixed feed PAOs may be produced according to U.S. Pat. No. 4,045,507 and 4,045,508 which disclose oligomerization processes that are useful with mixtures of 1-olefins as the feed, particularly mixtures of 1-decene with up to about 50 mole percent of 1-octene and/or 1-dodecene. Suitable processes include single reactor and multiple reactor schemes.

In a preferred embodiment, boron trifluoride is used as a catalyst together with a compound commonly called a promoter or a co-catalyst. The co-catalyst may be any compound known in the art which complexes with boron trifluoride to form a coordination compound which is catalytically active for the oligomerization reaction. Suitable co-catalysts include aliphatic ethers, such as dimethyl ether, diethyl ether and the like; aliphatic alcohols such as methanol, ethanol, n-butanol, decanol, and the like; polyols, such as ethylene glycol, glycerol and the like; water; aliphatic carboxylic acids such as acetic acid, propanoic acid, butyric acid, and the like; esters, such as ethyl acetate, methyl propionate, and the like; ketones, such as acetone and the like; aldehydes, such as acetaldehyde, benzaldehyde, and the like; acid anhydrides, such as acetic acid anhydride, succinic anhydride, and the like; and the like. It is preferred that the above-described co-catalyst comprise one to 10 carbon atoms although higher carbon co-catalyst compounds can be used. The co-catalyst can be used in a catalytic amount of 0.01 to about 3.0 weight percent, based on the amount of the olefins in the mixed olefin feed stream present, preferably from about 0.1 to about 1.0 weight percent.

The boron trifluoride will complex with the co-catalyst. It is preferred that the reaction liquid contain an excess of boron trifluoride in addition to that complexed with the co-catalyst in order to have a suitable rate of oligomerization. This may be accomplished by maintaining the pressure of boron trifluoride gas in the first reactor from about 5 to about 500 psig. (0.352 to 35.2 Kg/cm²) or higher with a preferred range of about 20 to about 100 psig. (1.41 to 7.03 Kg/cm²). Alternatively, the boron trifluoride gas can be bubbled through the liquid into the reactor maintained under atmospheric or higher pressure with boron trifluoride gas recycle. Other procedures are known in the art for introducing boron trifluoride into the reaction solution.

The temperature required for suitable oligomerization is conventional and can broadly range from about −20° to about 90° C., with a temperature in the range of about 200 to about 70° C. being preferred. The higher the temperature, the greater the rate of catalyst consumption and the lower the rate of reaction, while the lower the temperature, the greater the cooling costs with the reaction rate being satisfactory at the lower temperatures.

In an embodiment, a multiple reactor scheme is used wherein in the first-stage tank reactor, intimate mixing of the various components, the dissolution of boron trifluoride gas, and the control of the temperature of the reaction liquid during the exothermic oligomerization reaction is accomplished. The reactant mixed olefin feed stream and the catalysts are continuously introduced into the first stage tank reactor with reactor liquid being continuously withdrawn at a rate that will maintain a constant liquid level in the first reactor and fed into a second reactor. In order to provide a uniform distribution of the components in the first reactor it is desirable to provide some means to add the various components into the reactor and rapidly disperse them in order to provide a substantially homogeneous reaction medium. This can be conveniently accomplished by providing an externally driven stirrer. Another method involves the agitation of the entire reactor and contents by rocking or vibration, but this method is not particularly suitable with large reactors. Also bubbling boron trifluoride gas into the reactor may provide mixing of the reactor contents.

The expression tank reactor as used herein refers to a reactor in which the major dimensions are similar so that substantial homogeneity of composition and temperature can be maintained in the reaction fluid. Thus, it is preferred that the reactor itself be so dimensioned that the major dimension of the reactor differs from the minor dimension by a factor no greater than about ten to one, for example, height to diameter or vice versa. More preferably, it is desired that the major and minor dimensions of the reactor vary no more than by a factor of about five to one and most desirably by a factor no greater than about two and one-half to one for most effective functioning in accordance with the process herein. It is understood that the reactors will operate at a suitable liquid level for effective functioning thereof with stirring means preferred in each.

The exothermic oligomerization reaction is conveniently controlled in the first reactor by the diluting effect of the reactor liquid on the incoming mixed olefin feed stream. If greater dilution for temperature control is desirable, the reactor liquid volume can be increased, the inflow rate can be decreased, or an inert diluent can be added to the reactor with mixed olefin feed stream. Temperature control is also desirably accomplished by heat exchange means associated with the first reactor. Various factors which affect the degree of oligomerization taking place in this first stage reactor include the average time which the reaction liquid is in the reactor as measured by the liquid volume in the reactor divided by the flow rate from or into the reactor and the reaction rate, which is itself dependent on such factors as the concentration of the catalyst, the concentration of the mixed feed olefin stream, and the temperature.

In a multiple reactor scheme, a stream of reaction liquid is continuously removed from the first reactor and introduced into the second reactor. This reaction liquid contains oligomer, mixed olefin feed stream, boron trifluoride complex and boron trifluoride in solution. The oligomerization reaction proceeds further in this second reactor. Preferably the second tank reactor is provided with stirring means to insure homogeneity. If no additional tank reactors are to be used in this series, the degree of total overall conversion can be increased by increasing the ratio of reaction liquid in the second reactor to that in the first reactor. It is preferred in this series reactor arrangement, that the ratio of reactor volume in each downstream tank reactor to that in the first reactor be between about 1:5 and about 10:1, most preferably about 1:2 to about 5:1. When a series of three or more, generally up to a maximum of five or six tank reactors, is used, the reactors can conveniently be substantially identical in design. When only two reactors are used, it may be preferable to use a second tank reactor which is substantially greater in volume than the first reactor to more simply accomplish a greater proportion of the overall reaction in the second reactor. Although the temperature in the reactors can be the same or different, it is most practical to operate the reactors at the same or substantially similar temperature.

The relative amount of conversion undertaken in any of the reactors can be controlled by several techniques used alone or in combination. A convenient way, particularly in a relatively large reactor, is by raising or lowering the liquid level in the reactor without a substantial change in the flow-through rate. When the reactor liquid level (i.e., volume) is increased at constant inflow, the percent conversion in the tank reactor increases and when it is decreased, the percent conversion decreases. The conversion in a particular stage can also be controlled by changing the flow rate through the reactor at constant liquid volume, the higher the flow rate, the lower the conversion. This technique possesses the potential disadvantage that it changes the flow rate through the succeeding reactors and may affect the overall conversion particularly if the flow rate is increased. The percent conversion in any one of the reactors can also be controlled by reducing the reaction rate and this can be done by reducing the amount of co-catalyst, and consequently the amount of catalyst complex, below the optimum level. However, this technique may require the addition of co-catalyst to the input of other reactors for efficient utilization of the equipment.

Another method for controlling the conversion in the reactors involves the introduction of an inert diluent into the reactor. The inert diluent preferably is easily separated from the products and unreacted mixed olefin feed stream and can suitably be a hydrocarbon such as pentane, hexane, heptane and the like. This inert diluent technique is less preferred in the sense that the diluent must be separated out and it involves a less efficient use of the equipment. On the other hand, as noted above, an inert diluent can desirably help control the reaction temperature in a particular stage by its diluting effect. The inert diluent desirably is not used in an amount greater than 80 weight percent of the total reaction liquid and preferably no greater than about 50 weight percent of the total reaction liquid. However, the process may be carried out very effectively with no inert diluent.

In an embodiment, the mixed feed oligomer product is hydrogenated to stabilize it and protect it from oxidative degradation either before or subsequent to further processing to produce the mixed feed PAO of the present invention. Conventional hydrogenation catalysts such as palladium, platinum, nickel and the like at a suitable elevated temperature and pressure for hydrogenation, all of which are conventional in this field, are satisfactory. However, in another embodiment, the mixed feed oligomer product may not be hydrogenated, especially wherein the molecular weight of the oligomer product is sufficient to make hydrogenation unnecessary.

Cosmetic Formulations

Cosmetic formulations of the present invention may be solids, gels, soft solids, creams, lotions and/or particulate mixtures. These formulations can be applied using a range of different dispensers, including aerosol, roll-on, pump spray, sticks, and barrel dispensers, in accordance with the individual preferences of consumers. In an embodiment, the inventive cosmetic formulation is essentially free of volatile silicone oils, in particular, cyclomethicone tetramer, cyclomethicone pentamer, or both.

In an embodiment, the cosmetic formulation is a liquid, which is suitable for application from a propellant driven aerosol by use of a suitable propellant, examples of which are well known in the art. It should be noted that, when the composition is in the form of a liquid to be applied from a propellant driven aerosol, the percentages of the components of the composition referred to, in particular the amount of the cosmetic formulation in the composition, refer to the so-called “concentrate” composition, before its dilution with propellant and dosage into the aerosol container.

Propellant driven aerosol compositions according to the invention will be packaged with an aerosol propellant. The propellant gas can be any liquefiable gas known in the art for use in aerosol containers. Examples of suitable propellants include trichlorofluoromethane, trichlorotrifluoroethane, monochlorodifluorormethane, difluoroethane, propane, butane, isobutane, used singly or in combination. In such product forms, the composition according to the invention may typically comprise 5-45%, and the propellant 55-95% by weight of the total composition.

Particularly preferred product forms of compositions according to the invention are creams, lotions, gels and sticks. An alternative form is a lotion, which may conveniently be used in a roll ball dispenser, fitted with a ball valve, which may be applied to the skin in a conventional manner. A further possible composition would be a liquid suitable for dispensing from a finger operated pump spray or hand operated pump spray, which can deliver without the use of propellant gases a fine spray to the skin.

The composition can also take the form of a cream which is suitable or adapted for topical application to the skin. Depending on the product form, a number of other ingredients may be present in and form the balance of the topical compositions according to the invention. These may include:

Volatile and non volatile silicones, such as dimethyl cyclosiloxanes or polydimethyl siloxane, deoperfumes and deodorant compounds which can act as antimicrobial agents; minor amounts (for example less than about 10% by weight of the composition) of hydrophobic oils, such as liquid paraffin oils, isopropyl palmitate, and other emollients; thickening and gelling agents, such as clays, for example, Bentone 38, silicas, for example, Aerosil 200, organic waxes, such as castor wax, beeswax, or paraffin wax, silicone waxes, stearyl alcohol, fatty alcohols having 14-24 carbon atoms, fatty acids having 16-36 carbon atoms, propylene carbonate, and polyethylene; perfumes; preservatives and antioxidants; skin benefit agents, such as allantoin; humectants, such as polyols, for example glycerol; other cosmetic adjuncts conventionally employed in stick, roll on, propellant driven aerosol, pump spray and lotion products.

Particularly preferred optional components, in particular when the product form is a cream or lotion, include volatile silicones, which can preferably be present at a level of 40-60% by weight, propylene carbonate, which is preferably present at a level of 0.1-1% by weight, and silicas which are preferably present at a level of 2-8% by weight.

In a preferred embodiment, the cosmetic formulation is in a stick form, herein the term stick indicates a bar of semi-solid material which retains its integrity whilst being applied, i.e. a firm stick and which is commonly, though not exclusively, housed within a dispensing container which hitherto is conveniently in the shape of a barrel. When a portion of a firm stick is drawn across the skin surface, a film of the stick composition is transferred onto the skin surface. Although the stick has the appearance of a solid article, the material forming the stick usually comprises a structured liquid phase such that a film of the material is readily transferred onto another surface such as axillary skin upon contact under pressure.

The cosmetic formulations of the present invention may be in the form of suspension sticks, emulsion sticks and solution sticks. Suspension sticks contain a particulate biologically active material suspended in a structured carrier. Emulsion sticks normally comprise an emulsion of an oil phase and a hydrophilic phase containing the active material in solution, the continuous phase being structured. In some emulsion sticks, the continuous phase is an oil phase. In solution sticks, the active component is typically dissolved in the liquid carrier phase which is structured. The liquid phase can comprise water and/or a water-miscible organic solvent. The three categories can be applied to sticks of both firm and soft solids compositions.

The cosmetic formulations of the present invention may thus comprise naturally-occurring or synthetic waxes, of which typical examples include stearyl alcohol, hydrocarbon waxes, waxes of plant or animal origin or their synthetic analogues or derivatives or silicone waxes. Waxes are widely available, and by suitable selection of the waxes themselves and their concentrations in the formulation can effectively obtain either a soft solid or a firm solid.

It is highly desirable to employ components which impart little or no color to the inventive cosmetic formulations. Accordingly, it is preferred to avoid or minimize the incorporation of alkoxy cinnamates and other highly colored components unless the particular color is desired. In addition, it is desirable to avoid employing or at least minimize the presence of any component which has an intrinsically high and unpleasant odor, or one which would cause unacceptable irritation.

The cosmetic formulation may comprise at least 5 wt % mixed feed PAO. In a preferred embodiment, the cosmetic formulation may comprise at least 10 wt %, preferably at least 20 wt %, preferably at least 30 wt %, preferably at least 40 wt %, preferably at least 50 wt %, preferably at least 60 wt %, preferably at least 70 wt %, preferably at least 80 wt %, preferably at least 90 wt % of the mixed feed PAO.

In addition to the mixed feed PAO, the cosmetic formulations may comprise liquid aliphatic hydrocarbons such as mineral oils or hydrogenated polyisobutene, often selected to exhibit a low viscosity. Conventional poly alpha olefins such as polydecene may also be used, as may paraffins and isoparaffins of at least 10 carbon atoms. The components of the cosmetic formulation are preferably selected to achieve a refractive index of greater than 1.4, preferably greater than 1.45, more preferably greater than 1.46.

The cosmetic formulation may also include oxygen containing emollients, such as alkyl benzoate, alkylene dibenzoate, alkoxylated alkyl benzoate or a polyalkylene oxide dibenzoate, or a mixture of two or more thereof. The alkyl group often contains at least 10 carbons, in many instances up to 25 carbons and may be linear and/or branched. Preferred alkyl groups are in the range of from 12 to 20 carbons and include dodecyl (lauryl) terdecyl, tetradecyl (myristyl), pentadecy, hexadecyl (palmityl), octdecyl (stearyl) 2-methyl-heptadecyl (iso-stearyl) and octyldodecyl groups. A mixture of two or more of the alkyl groups can be employed, such as a mixture of C₁₂-C₁₅ alkyl groups. The term alkylated herein includes alkylene groups and the latter are terminated at each end with a benzoate group. The alkylene group often contains from 2 to 6 carbons and can be linear or branched, e.g., propylene.

Alkoxylated alkyl benzoates contemplated for use herein include an alkyl group terminated by an alkoxy group, which can be monomeric containing for example up to 6 carbons or polymeric such as polyethylene oxide or preferably polypropylene oxide, which conveniently comprises up to 30 units and often from 5 to 20 units. The alkyl group can be selected from the previously identified alkyl groups. Alternatively, the benzoate compound can comprise a polyethylene oxide or polypropylene oxide moiety, or preferably a block copolymer of ethylene oxide and propylene oxide, terminated at each end by a benzoate group. Mixtures of two or more of the above benzoates can be employed. Preferably, these components have a refractive index in the region of 1.465 to 1.49.

The cosmetic formulation may also comprise volatile and/or non-volatile silicone oils. Such compounds commonly comprise alkylphenyl substituted polysiloxanes, and especially methylphenyl polysiloxanes. Polysiloxane is preferably short chain and linear, such as a disiloxane, trisiloxane or tetrasiloxane. A mole ratio of alkyl (especially methyl) to phenyl substitution of 1:1 is preferred. In an embodiment, non-volatile polysiloxane materials preferably include those which have a viscosity of below 300 centistokes (300×10⁻⁶ M²s⁻¹) with below 200 centistokes being more preferred. Preferably, the viscosity of the siloxane materials may also be 50 centistokes or higher. The refractive index of preferred non-volatile silicone oils, such as those comprising alkylphenylsiloxanes is preferably 1.50 to 1.56.

The cosmetic formulation may also comprise other fluids which are miscible with the mixture of components, or are soluble in the final combination, thereby forming an anhydrous fluid carrier. Anhydrous herein indicates that the formulation is free from a distinct aqueous phase, which means in practice that it does not comprise an aqueous emulsion or micro-emulsion. The choice of other carrier fluids is at the discretion of the formulator, within the bounds indicated herein. In practice, they tend to be hydrophobic, although a limited proportion of hydrophilic constituents can be employed, such as those materials which, in the chosen proportions, are still miscible with the remainder of the carrier fluid in the cosmetic formulation.

The cosmetic formulation may further comprise volatile liquid silicones, i.e. liquid polyorganosiloxanes. To class as “volatile” such material should have a measurable vapor pressure at 20 or 25° C. The vapor pressure of a volatile silicone is preferably from 1 or 10 Pa to 2 kPa at 25° C.

It can be desirable for the cosmetic formulation to include a volatile silicone because it gives a “drier” feel to the applied film after the composition is applied to skin. The carrier fluid often contains from 0 to 50% and particularly from 10 to 40% by weight volatile silicone.

Volatile polyorganosiloxanes can be linear or cyclic or mixtures thereof. Preferred cyclic siloxanes include polydimethylsiloxanes and particularly those containing from 3 to 9 silicon atoms and preferably not more than 7 silicon atoms and most preferably from 4 to 6 silicon atoms, otherwise often referred to as cyclomethicones. Preferred linear siloxanes include polydimethylsiloxanes containing from 3 to 9 silicon atoms. The volatile siloxanes normally by themselves exhibit viscosities of below 10⁻⁵ m²/sec (10 centistokes), and particularly above 10⁻⁷ m²/sec (0.1 centistokes), the linear siloxanes normally exhibiting a viscosity of below 5×10⁻⁶ m²/sec (5 centistokes). The volatile silicones can also comprise branched linear or cyclic siloxanes such as the aforementioned linear or cyclic siloxanes substituted by one or more pendant —O—Si(CH₃)₃ groups.

The cosmetic formulation may also comprise liquid aliphatic esters such as aliphatic esters having at least one long chain alkyl group. Preferred aliphatic esters are derived from C₁ to C₂₀ alkanols esterified with a C₈ to C₂₂ alkanoic acid or C₆ to C₁₀ alkanedioic acid. The alkanol and acid moieties or mixtures thereof are preferably selected such that they each have a melting point of below 20° C. These esters include isopropyl myristate, lauryl myristate, isopropyl palmitate, diisopropyl sebacate and diisopropyl adipate. The proportion of aliphatic esters in the carrier fluid is often chosen within the range of 0 to 50% and particularly 0 to 25% of the carrier.

The cosmetic formulation may also comprise aliphatic alcohols, preferably those which are liquid at 20° C. These include branched chain alcohols of at least 2, and preferably at least 10 carbon atoms e.g. 10 to 25 carbons, such as isostearyl alcohol and octyl dodecanol. The proportion of liquid aliphatic alcohol in the carrier fluid is often chosen within the range of 0 to 50% and particularly 0 to 25% of the carrier.

Other suitable components of the cosmetic formulation include aliphatic ethers that are liquid at 20° C., which are derivable from at least one alkanol containing at least 4 carbons and often up to 18 carbons, and which often contain a polyalkylene glycol moiety. Examples of such ethers include myristyl ether derivatives e.g. PPG-3 myristyl ether or lower alkyl ethers of polypropylene glycols such as PPG-14 butyl ether. The proportion of liquid aliphatic ethers in the carrier fluid is often chosen within the range of 0 to 50% and particularly 0 to 25% of the carrier.

Structurants

The cosmetic formulation may also comprise a structurant to provide physical rigidity to the composition. Structurants include waxes and gellants.

The term “wax” is conventionally applied to a variety of materials and mixtures which have similar physical properties, namely that:
they are solid at 30° C. and preferably also at 40° C.;
they melt to a mobile liquid at a temperature above 30° C. but generally below 95° C. and preferably in a temperature range of 40° C. to 90° C.;
they are water-insoluble and remain water-immiscible when heated above their melting point.

Waxes are usually hydrocarbons, or silicone polymers, or linear fatty alcohols, esters of fatty acids or glyceride derivatives or mixtures containing such compounds, possibly also containing a minority (less than 50%) of other compounds. Naturally occurring waxes are often mixtures of compounds which include a substantial proportion, likely to be a majority, of fatty esters.

A wax forms crystals in the carrier fluid when it cools from the heated state during processing. These crystals take various forms including needles and platelets depending on the individual waxes. Some waxes form a network of fibrous crystals and can therefore also be identified as fiber-forming structurants.

Examples of hydrocarbon waxes include paraffin wax, microcrystalline wax and polyethylenes with molecular weight of 2,000 to 25,000. Waxy linear fatty (aliphatic) alcohols normally contain at least 10 and preferably at least 12 carbon atoms, in practice often not more than 40 carbon atoms and many preferred alcohols contain from 14 to 25 carbon atoms.

Two suitable classes of ester waxes include glycerol or glycol esters and alkyl alkanoate esters. The waxes are selected from fatty acid derivatives of glycerol or glycol, such as ethylene glycol. Preferably at least two ester groups are present in the ester waxes. The fatty acid moiety therein normally contains at least 10 carbons and especially from 12 to 24 carbons. Commonly the esters are derived from stearic acid or benhenic acid or a mixture of fatty acids, such as those containing either or both of said acids e.g. C₁₆-C₂₂ fatty acids. It is especially desired to employ glycerol esters. Some esters may be present as components in certain naturally occurring waxes and they these may also be made synthetically. A second class of ester wax comprises esters which have a melting point of at least 30° C. and which satisfy the general formula below:
CH₃—(CH₂)ₙ—O—CO—(CH₂)ₘ—CH₃
in which n is from 9 to 39 and m is from 0 to 35. Within this general formula, a range of preferred esters comprises those in which n is selected within the range of 14 to 24 and especially 16-22 together with m being selected in the range of 14 to 24 and especially 16 to 22. In second range of preferred esters within the general formula, n is selected in the range of 18 to 38 and m is either 0 or 1. It will be understood that mixtures of esters within each preferred range or mixtures of one preferred range of esters with the other can be employed. Some convenient mixtures include a mixture of a wax comprising esters of n=14 to 20 and m=14 to 20 with a wax comprising esters of n=16 to 20 and m=14 to 20 or preferably 16 to 20. Mixtures of the ester waxes can be employed, either within either class of ester or a mixture of both classes.

A useful class of waxes comprises those which comprise or consist of glyceride waxes and in particular triglyceride waxes. Many suitable glyceride waxes comprise esters of fatty acids, often containing at least 16 carbon atoms, and especially from 18 to 36 carbon atoms. Examples of suitable ester moieties include stearate, eicosinate and behenate. Certain of them can be derived from naturally occurring oils such as castor oil by hydrogenation. Yet others include tristearin, or are obtainable by hydrogenating vegetable oils such as rape seed oil. A number of triglyceride waxes are obtainable from Croda Chemicals under their trade name Syncrowax, e.g. grades HRC and HGL-C.

Examples of natural waxes or simple derivatives of natural products include castor wax, beeswax, carnauba and candelilla waxes, which are of vegetable origin and mineral waxes from fossil remains other than petroleum. Montan wax, which is an example of mineral wax, includes non-glyceride esters of carboxylic acids, hydrocarbons and other constituents. Other naturally available waxes include spermeceti wax, ozokerite, ceresin, baysberry, and synthetic waxes such as Fisher-Tropsch waxes and microcrystalline waxes.

Waxes useful in the present invention will generally be those found to thicken water-immiscible oxygen-containing oils such as C₁₂₋₁₅ alkyl benzoates and/or non-volatile methylphenylpolysiloxanes, when dissolved therein (by heating and cooling) at a concentration of 5 to 25% by weight.

The wax is normally employed in such an amount that the carrier fluid is structured in combination with any other structurant that is present and the presence of any particulate antiperspirant or other biologically active material. This amount is usually not greater than the weight of the carrier fluid, and in most instances not greater than 30% of the weight of the cosmetic formulation.

If a wax is used which forms a network of fibers, the amount of it may commonly be from 4 to 12% by weight of the composition. If a wax is used which does not form such a network, for instance a wax which crystallizes as spherulitic needles or as small platelets, the amount is often selected in the range of from 4 to 25% and in many preferred embodiments from 5 to 12% or 10 to 25% of the composition, depending at least in part upon whether the wax is being employed in conjunction with or without a further structurant. Silicone waxes are an example of waxes which crystallize as small platelets.

A combination of waxes may also be used in the present cosmetic formulation. Preferred combinations include a combination of a glyceride wax with at least one additional wax selected from glycerol ester waxes, alkylalkanoate waxes and fatty alcohols. Preferably, the glyceride wax is present in a weight ratio to the other wax or waxes of 1:2 to 1:6 and more preferably from 2:5 to 1:4.

Fiber-Forming Gellants

In an embodiment, the cosmetic formulation of the present invention may comprise a gellant. A number of organic compounds are known to possess the ability to gel water-immiscible organic liquids such as water-immiscible hydrocarbon and/or silicone oils. Such materials are generally non-polymeric, i.e. monomers or dimers with molecular weight below 10,000 often below 5,000 or even 1,000 rather than polymers with more than four repeat units or with molecular weight above 10,000.

Gel formation takes place as an exothermic event within a temperature range referred to as the gel point; upon re-heating, melting of the gel takes place as an endothermic event within a temperature range. Such gels can be disrupted by shearing. Although a small partial recovery may then be observed, such gels do not recover their structure for a long time, if at all, unless re-melted.

It is characteristic of such non-polymeric gellants (structurants), useful in this invention, that they are able to gel the organic liquid in the absence of any disperse phase, when used in sufficient quantity not exceeding 15% by weight; the structured liquids are obtainable by cooling from an elevated temperature at which the structurant is in solution in the liquid—this hot solution being mobile and pourable; the (thus obtained) structured liquid becomes more mobile if subjected to shear or stress; the structure does not spontaneously recover within 24 hours if the sheared liquid is left to stand at ambient laboratory temperature, even though a small partial recovery may be observed; and the structure can be recovered by re-heating to a temperature at which the structurant is in solution in the liquid and allowing it to cool back to ambient laboratory temperature.

It appears that such non-polymeric structurants operate by interactions which are permanent unless disrupted by shear or heating. Such structurants form a network of strands or fibers extending throughout the gelled liquid. In some cases these fibers can be observed by electron microscopy, although in other cases the observation of the fibers which are believed to be present is prevented by practical difficulties in preparing a suitable specimen. When observed, the primary fibers in a gel are generally thin (diameter less than 0.5 micrometers, often less than 0.2 micrometers) and appear to have numerous branches or interconnections. Primary fibers may entwine to form a thicker strand.

If these fibers are crystalline, they may or may not be the same polymorph as macroscopic crystals obtained by conventional crystallization from a solvent. One material suitable for use herein which is well known to form such gels is 12-hydroxy stearic acid (12-HAS.) In addition, U.S. Pat. No. 5,750,096 is one of several references directed to gelation brought about using esters or amides of 12-hydroxy stearic acid. The alcohol used to form such an ester or the amine used to form such an amide may contain an aliphatic, cycloaliphatic or aromatic group with up to 22 carbons therein. If the group is aliphatic it preferably contains at least three carbon atoms. A cycloaliphatic group preferably contains at least five carbon atoms and may be a fixed ring system such as adamantyl. Other fatty acids with C₈ or longer alkyl chains may be used and amides thereof can also be used. A specific example is lauric monoethanolamide also termed MEA lauramide.

N-acyl amino acid amides and esters may also be employed herein to structure liquids, preferably by forming fibrous networks. Examples of suitable N-acyl amino acid amides and esters include N-α,N-δ,dicaprylylornithine octyl, decyl, lauryl and stearyl ester, N-α,N-ε,-dilauroyllysine hexyl, octyl, decyl, and lauryl esters, N-α,N-ε,-di(tallowyl) and N-α,N-ε,-di(hydrogenated tallowyl) lysine hexyl, octyl, decyl, and lauryl esters, in which tallowyl indicates the acyl radical of tallow fatty acid.

Examples of N-acylamino acid amides include N-acetyl glutamic acid-α,γ-dilauryl and α,γ-distearyl amides; N-lauroyl glutamic acid diamide, -α,γ-dibutyl, -α,γ-dihexyl-, -α,γ-dioctyl, -α,γ-dilauryl and -α,γ-distearyl amides; N-cocoyl glutamic acid, -α,γ-diamide, -α,γ-dibutyl, -α,γ-dihexyl, -α,γ-dioctyl, -α,γ-dilauryl and -α,γ-distearyl amides; N-hydrogenated tallowyl glutamic acid, -α,yγ-diamide, -α,γ-dibutyl, -α,γ-dihexyl, -α,γ-dioctyl, -α,γ-dilauryl and -α,γ-distearyl amides; N-α,N-ε,-dicaproyllysine amide, butyl hexyl, octyl, lauryl, and stearyl amides; N-α,N-ε,-dicapryloyllysine amide, butyl, dibutyl, hexyl, octyl, lauryl, and stearyl amides; N-α,N-ε,-dilauroyllysine amide, butyl, hexyl, octyl, lauryl, and stearyl amides; N-α,N-ε,-dicocoyllysine amide, butyl hexyl, octyl, lauryl, and stearyl amides; N-α,N-ε,-di(hydrogenated tallowyl) lysine amide, butyl hexyl, octyl, lauryl, and stearyl amides; N-lauroylvaline amide, butyl, hexyl, octyl and lauryl amides; N-lauroyl-phenylalanine amide, butyl, hexyl, octyl and lauryl amides; N-capryloyl leucine amide, butyl, hexyl, octyl and lauryl amides; and N-palmitoylmethionine amide, butyl, hexyl and octyl amides.

Further materials which have been disclosed as gelling agents are the amide derivatives of di and tribasic carboxylic acids set forth in WO 98/27954 notably alkyl N,N′ dialkyl succinamides. Lanosterol, as disclosed in U.S. Pat. No. 5,635,165 mentioned above may suitably be used if the water-immiscible fluid comprises a major fraction of silicone oils. In a preferred embodiment, β-sitosterol or campesterol or cholesterol, or a hydrogenated derivative thereof, such as dihydrocholesterol, or a mixture of two or more of them may be used. An especially preferred sterol is β-sitosterol. A preferred sterol ester is oryzanol, sometimes referred to as γ-oryzanol. The sterol and sterol ester are used in a mole ratio that is normally selected in the range of from 10:1 to 1:10, especially from 6:1 to 1:4 and preferably in the range of from 3:1 to 1:2. Employment of the two system constituents within such a mole ratio range, and especially within the preferred range facilitates the co-stacking of the constituents and consequently facilitates the formation of a network that is readily able to structure the cosmetic formulation.

Biologically Active Components

The cosmetic formulation of the present invention may also comprise one or more biologically active components. Biologically active components include materials having known biological activity, such as sunscreens, antiperspirants, deodorants, analgesic actives, and the like. Preferably, the biologically active components are suitable for being topically applied in a cosmetic formulation.

Sunscreens

A wide variety of conventional sunscreening agents suitable for use in the present invention include, for example: p-aminobenzoic acid, its salts and its derivatives (ethyl, isobutyl, glyceryl esters; p-dimethylaminobenzoic acid); anthranilates (i.e., o-aminobenzoates; methyl, menthyl, phenyl, benzyl, phenylethyl, linalyl, terpinyl, and cyclohexenyl esters); salicylates (octyl, amyl, phenyl, benzyl, menthyl, glyceryl, and dipropyleneglycol esters); cinnamic acid derivatives (menthyl and benzyl esters, -phenyl cinnamonitrile; butyl cinnamoyl pyruvate); dihydroxycinnamic acid derivatives (umbelliferone, methylumbelliferone, methylaceto-umbelliferone); trihydroxycinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol13,6-disulfonic and of 2-naphthol-6,8-disulfonic acids); dihydroxy-naphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and vilouric acids; tannic acid and its derivatives (e.g., hexaethylether); (butyl carbityl) (6-propyl piperonyl) ether; hydroquinone; benzophenones (oxybenzene, sulisbenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; and 4-isopropyl-di-benzoylmethane.

Of these, 2-ethylhexyl p-methoxycinnamate, 4,4′-t-butyl methoxydibenzoylmethane, 2-hydroxy-4-methoxybenzophenone, octyldimethyl p-aminobenzoic acid, digalloyltrioleate, 2,2-dihydroxy-4-methoxybenzophenone, ethyl 4-[bis(hydroxypropyl)]aminobenzoate, 2-ethylhexyl 12-cyano-3,3-diphenylacrylate, 2-ethylhexysalicylate, glyceryl p-aminobenzoate, 3,3,5-trimethylcyclohexysalicylate, methylanthranilate, p-dimethyl-aminobenzoic acid or aminobenzoate, 2-ethylhexyl p-dimethylamino-benzoate, 2-phenylbenzimidazole-5-sulfonic acid 2-(p-dimethylaminophenyl)-5-sulfonicbenzoxazoic acid and mixtures of these compounds, are particularly preferred.

Preferred sunscreens useful in the compositions of the present invention also include 2-ethylhexyl p-methoxycinnamate, butylmethoxydibenzoylmethane, 2-hydroxy-4-methoxybenzophenone, octyldimethyl p-aminobenzoic acid, 2,2′dihydroxy-4-methoxybenzophenone and ethyl hexyl salicylate and mixtures thereof.

A safe and photoprotectively effective amount of sunscreen may be used in the sunscreen compositions of the present invention. By “safe and photoprotectively” is meant an amount sufficient to provide photoprotection when the composition is applied not so much as to cause any side effects or skin reactions. Generally from about 1% to about 30%, preferably from about 2% to about 20%, of the composition may comprise a sunscreening agent. Exact amounts will vary depending upon the sunscreen chosen and the desired Sun Protection Factor (SPF).

SPF is a commonly used measure of photoprotection of a sunscreen against erythema. This number is derived from another parameter, the minimal erythemal dose (MED). MED is defined as the “lease exposure dose at a specified wavelength that will elicit a delayed erythema response”. The MED indicates the amount of energy reaching the skin and the responsiveness of the skin to the radiation. The SPF of a particular photoprotector is obtained by dividing the MED of protected skin by the MED of unprotected skin. The higher the SPF, the more effective the agent in preventing sunburn. The SPF value tells how many times longer a person can stay in the sun with use of the sunscreen (compared to a person with unprotected skin) before that person will experience 1 MED. For example, utilizing a sunscreen with an SPF of 6 will allow an individual to stay in the sun six times longer before receiving 1 MED. As the SPF value of a sunscreen increases, the less chance exists for development of tanning of the skin. Commercially available sunscreening products have SPF values ranging from 2 to 50.

Also particularly useful in the present invention are sunscreens having, in a single molecule, two distinct chromophore moieties which exhibit different ultra-violet radiation absorption spectra. One of the chromophore moieties absorbs predominantly in the UVB radiation range and the other absorbs strongly in the UVA radiation range. These sunscreening agents provide higher efficacy, broader UV absorption, lower skin penetration and longer lasting efficacy relative to conventional sunscreens.

Preferred members of this class of sunscreening agents are 4-N,N-(2-ethylhexyl)methylaminobenzoic acid ester of 2,4-dihydroxybenzophenone; N,N-di-(2-ethylhexyl)-4-aminobenzoic acid ester with 4-hydroxydibenzoylmethane; 4-N,N(2-ethylhexyl) methylaminobenzoic acid ester with 4-hydroxydibenzoylmethane; 4-N,N-(2-ethylhexyl)methylaminobenzoic acid ester of 2-hydroxy-4-(2-hydroxyethoxy)benzophenone; 4-N,N-(2-ethylhexyl)methylaminobenzoic acid ester of 4-(2-hydroxyethoxy)dibenzoylmethane; N-N-di-(2-ethylhexyl)-4-aminobenzoic acid ester of 2-hydroxy-4-(2-hydroxyethoxy)benzophenone, and N,N-di-(2-ethylhexyl)4-aminobenzoic acid ester of 4-(2-hydroxyethoxy)dibenzoylmethane and mixtures thereof.

An agent may also be added to any of the compositions of the present invention to improve the skin substantively of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157, which is incorporated herein by reference. The disclosed skin substantively agent comprises the polymeric form of two monomers, ethylene and acrylic acid. These copolymers are preferably included in an oil-in-water emulsion sunscreen composition comprising: (a) from about 1% to about 20% of the chelating agent plus an optional oil-soluble sunscreen; (b) from about 0.25% to about 3% of the ethylene-acrylic acid copolymer as described above; (c) from about 2% to about 10% of an emulsifier; and (d) from about 70% to about 90% of water, wherein the ratio of photo protecting agents to the copolymer is from about 12:1 to about 15:1. Sunscreening agents which are particularly useful in combination with these copolymers are 2-ethylhexyl p-methoxycinnamate, butyl methoxydibenzoylmethane, 2-hydroxy-4-methoxybenzophenone, octyldimethyl p-aminobenzoic acid and mixtures thereof.

Antiperspirant Actives

Preferred antiperspirant actives useful herein include the following: polyhydroxy complexes of basic aluminum salts as described in U.S. Pat. Nos. 3,420,932; 3,359,169; 3,523,130; 3,507,896; 3,873,686; 3,876,758; 3,555,146; Britain Patent 1,159,685; and 1,159,686.

Zirconyl hydroxychloride salts, especially zirconium-aluminum-glycine complexes (“ZAG complexes”) useful herein include those described in Belgium Patent Specification No. 825,146; U.S. Pat. Nos. 2,814,585, 3,679,068; 4,017,599; 4,120,948; and Britain Patent Specification No. 2,144,992.

Also useful are Aluminum chlorhydroxide (“ACH”) salts as described in U.S. Pat. Nos. 3,887,692; 3,904,741; 4,359,456; Britain Patent Specification No. 2,048,229; and 1,347,950.

Aluminum chlorhydroxide salts, zirconyl hydroxychloride salts, and mixtures thereof having improved molecular distributions are known, having been disclosed, for example, in U.S. Pat. No. 4,359,456 and European Patent Application Publication Nos. 6,739; 183,171; 191,628; and British Patent Specification Nos. 2,048,229; 2,144,992. Antiperspirant actives with enhanced efficacy due to improved molecular distribution are also described in European Patent Application Publication No. 7,191.

The antiperspirant active typically comprise in total from about 1% to about 40%, preferably from about 10% to about 30%, and most preferably from about 10% to about 25%, of the cosmetic compositions of the present invention.

Analgesic Actives

Preferred analgesic actives suitable for use in the cosmetic formulation of the present invention include methyl salicylate, turpentine oil, menthol, camphor, histamine dihydrochloride, methyl nicotinate, eucalyptus oil, triethanolamine salicylate, glycol salicylate and salicylamide and mixtures thereof. Suitable analgesic actives are fully described in “Handbook of Nonprescription Drugs”, published by The American Pharmaceutical Association, seventh edition (1982) pp 514-523 which is incorporated by reference herein. The analgesic agent typically comprises from about 0.1% to about 40% by weight of the composition.

Accordingly, the composition of the present invention is also contemplated for medicinal compositions and use.

Optional Ingredients

In addition to the ingredients listed above, the cosmetic formulation of the present invention can include other optional ingredients that are conventionally included in topically applied cosmetic compositions. Optional ingredients in compositions of this invention can include disinfectants, for example at a concentration of up to about 10% w/w. Suitable deodorant actives can comprise deodorant effective concentrations of antiperspirant metal salts, deoperfumes, and/or microbicides, including particularly bactericides, such as chlorinated aromatics, including biguanide derivatives, of which materials known as triclosan (Irgasan DP300™), chlorhexidine and Tricloban™, warrant specific mention. A yet another class comprises biguanide salts such as available under the trade mark CoSmosil™.

The cosmetic formulation may further comprise one or more wash-off aids, often in a proportion of up to about 10% by weight, especially up to about 5% by weight and particularly from 0.5 to 3% by weight based on the formulation.

Suitable examples of wash off aids commonly comprise nonionic surfactants and especially nonionic surfactants which contain a polyalkylene oxide moiety, the residue of a fatty acid or fatty alcohol and optionally the residue of an aliphatic polyhydric alcohol linking group. Although, the surfactants may comprise a single fatty residue, they preferably contain two residues. Preferably, the surfactant is an ester surfactant, and especially a diester surfactant. The polyalkylene oxide is often polyethylene oxide, or polypropylene oxide or mixed polyethylene oxide/propylene oxide, the polymer containing from 3 to 50 and especially from 5 to 20 alkylene oxide units. The fatty acid or alcohol often contains from 12 to 24 carbons, and in many instances is linear, examples including 16, 18 or 22 linear carbons. Especially preferred wash-off aids herein comprise polyethylene oxide diesters of fatty alcohols containing 16 to 22 linear carbons, such as PEG-8 distearate.

One important class of optional constituents comprises fragrances. They can be incorporated into the cosmetic formulations in an amount of from 0% to about 5% and often from 0.2 to 1.5% by weight based on the total weight of the composition. Fragrance-containing compositions of the present invention, when applied to skin, fix a substantive fragrance film on the skin that resists moisture, but that can be removed by washing.

One other class of ingredients comprises moisturizing agents such as humectants. These include propylene glycol, sorbitol and especially glycerol. Moisturising agents often comprise from 0 to 5% by weight of the formulation, and if employed, it is desirable that the amount is chosen such that the agent is retained within the carrier fluid.

Yet other optional ingredients that can be included in the anhydrous composition of the present invention include, but are not limited to, drying agents, like talc or DRY FLO (aluminum starch octenylsuccinate); preservatives; and dyes. Generally, such optional ingredients are present in a composition of the present invention in an amount of about 10% or less by weight. In addition, an organoclay can be included in a composition of the present invention as an additional suspending agent in an amount of up to 20% by weight of the composition. An organoclay is potentially helpful as an anti-caking agent to maintain a particulate topically-effective compound homogeneously dispersed throughout the composition. An exemplary organoclay is a quaternised three-layer clay exfoliated with a polar solvent, like a quaternised montmorillonite clay exfoliated with propylene carbonate. Such clays are available under the trade name “Bentone”. Other inorganic materials which can be incorporated, for example as a thickener for the formulation, comprise particulate silica, such as fumed silica, suitably in an amount of up to 5%. Still other cosmetic adjuncts can include:

Skin feel improvers, such as talc (mentioned hereinabove) or finely divided polyethylene or glyceryl fatty esters, e.g. glyceryl stearate, incorporated, for example, in an amount of up to about 10% w/w; Skin benefit agents such as allantoin or lipids, for example in an amount of up to 5% w/w; and skin cooling agents, such as menthol and menthol derivatives, often in an amount of up to 2% w/w.

Stick formulations produced in accordance with the present invention may be opaque. However, it is possible to obtain sticks having an appearance which is at least slightly translucent, for example by so selecting the constituents of the carrier fluid and the nature of the antiperspirant active that the refractive index of the carrier fluid and dispersed solid differs by no more than about 0.06, preferably employing antiperspirant active particulates which lack substantial internal voids. Substantially internal void-free actives can be obtained by milling void-containing actives.

Whilst the ingredients disclosed herein are effective for preparing the cosmetic formulations of the instant invention, prospective manufacturers should keep abreast of advances in scientific understanding of their effect on humans and particularly on skin before selling the resultant compositions for topical application to skin.

In an embodiment, the present invention includes a method of making a composition for topical application to human skin, comprising a biologically active material and a cosmetically suitable carrier, comprising combining the inventive mixed feed polyolefin into a carrier to produce a cosmetically suitable carrier. Preferably, the cosmetically suitable carrier comprises 5-95 wt % of the mixed feed polyolefin based on the total weight of the composition. Also preferably, the feed polyolefin comprises two or more C₆-C₂₀ copolymerized monomers. In a preferred embodiment, at least two of the copolymerized monomers are each present in the mixed feed polyolefin at 10 wt % or more, based on the total weight of the mixed feed polyolefin.

The compositions described herein can be produced by conventional processes for making the various forms of cosmetic formulations.

A convenient process sequence for suspension antiperspirant formulations comprises first mixing the structurant or mixture of structurants, namely the wax(es), the non-polymeric fiber-forming gellant(s) or a mixture of both with the carrier at a temperature that is high enough to melt the structurant. Thereafter, particulate antiperspirant active can be blended with the carrier solution and the blend is formed into a solid mass by cooling, for example by being introduced into its dispensing container at a temperature that is often 5 to 10° C. above its normal setting temperature. The process normally includes a suitable filling process, such as a pour fill process (sometimes gravity-fed injection) or injection at elevated pressure into a dispensing container such as a barrel where it is cooled or allowed to cool to ambient.

The compositions herein are suitable for applying topically to human skin, and particularly antiperspirant compositions to axillae, thereby reducing observable perspiration.

Product Packages

The compositions of this invention include those which are structured liquids having a firm appearance. Such a composition of this invention will usually be marketed as a product comprising a container with a quantity of the composition therein, where the container has an aperture for the delivery of composition, and means for urging the composition in the container towards the delivery aperture. Conventional containers take the form of a barrel of oval cross section with the delivery aperture(s) at one end of the barrel. An alternative cross section is round.

In an embodiment, a composition of the present invention is typically sufficiently rigid that it is not apparently deformable by hand pressure. It is suitable for use as a stick product in which a quantity of the composition in the form of a stick is accommodated within a container barrel having an open end at which an end portion of the stick of composition is exposed for use. The opposite end of the barrel is closed.

Generally the container will include a cap to cover its open end and a component part which is sometimes referred to as an elevator or piston fitting within the barrel and capable of relative axial movement along it. In such packaging, the stick of composition is accommodated in the barrel between the piston and the open end of the barrel. The piston is used to urge the stick of composition along the barrel. The piston and stick of composition may be moved axially along the barrel by manual pressure on the underside of the piston using a finger or rod inserted within the barrel. Another possibility is that a rod attached to the piston projects through a slot or slots in the barrel and is used to move the piston and stick. Preferably the container also includes a transport mechanism for moving the piston. One transport mechanism comprises a threaded rod which extends axially into the stick through a correspondingly threaded aperture in the piston, and means mounted on the barrel for rotating the rod. Conveniently, the rod is rotated by means of a hand-wheel mounted on the barrel at its closed end, i.e. the opposite end to the delivery opening. The component parts of such containers are often made from thermoplastic materials, for example polypropylene or polyethylene.

### EXAMPLES

Having described the invention in general terms, specific embodiments thereof will now be described in some detail by way of example only.

| | | |
|---|---|---|
| | INCI Designation/ | |
| Component | Description | Source |

Listing of Components

| | | |
|---|---|---|
| PPG-14 Butyl | Diluent/carrier | Croda |
| Ether | | |
| Carbopol 980 | Carbomer | Noveon |
| (2% solution) | | |
| Hydrogenated | Diluent/carrier | ACME-Hardesty |
| Castor Oil | | |
| PEG-8 Distearate | Surfactant | Stepan |
| Stearyl Alcohol | Diluent | Croda |
| Disodium EDTA | Chelating agent | BASF |
| Propylene Glycol | Diluent/carrier | Arch Chemical |
| Promulgen D | Cetearyl alcohol/ | Amerchol |
| | Ceteareth-20 | |
| Dow Corning | Cyclomethicone | Dow Corning |
| 245 Fluid | | |
| Dow Corning | Cyclomethicone | Dow Corning |
| 345 Fluid | | |
| Dow Corning | PPG/PEG-18/18 | Dow Corning |
| 190 surfactant | Dimethicone | |
| Bentone Gel | Cyclomethicone/ | Rheox |
| VS-5 PC | Quaternium-18/ | |
| | Hectorite/propylene | |
| | carbonate | |
| Rezal 36GP | Aluminum Zirconium | Reheis |
| | Tetrachlorohydrex-GLY | |
| Cabosil M-5 | Silica | Degussa |
| Talc 127 | Talc | MPSI |
| Talc 5251L | Talc | MPSI |
| Reach AZP | Aluminum Zirconium | Reheis |
| 908 SUF | Tetrachlorohydrex-GLY | |
| Fragrance | | |
| Arlacel 165 | Glyceryl Stearate | Uniqema |
| | (and) PEG-100 Stearate | |
| Triethanolamine | pH adjustment | Dow Chemical |
| Germaben II | preservative | Sutton Laboratories |

Comparative PAO

| | | |
|---|---|---|
| PureSyn 2 | Comparative | ExxonMobil |
| | 2 cSt Polydecene | |
| | (C₁₀) PAO | |
| PureSyn 4 | 4 cSt Polydecene | ExxonMobil |
| | (C₁₀) PAO | |
| PureSyn 6 | 6 cSt Polydecene | ExxonMobil |
| | (C₁₀) PAO | |
| SpectraSyn | 6cSt Polydodecene | ExxonMobil |
| 6 LN | (C₁₂) PAO | |
| SpectraSyn | 40 cSt C₁₀/C₁₂ Blend | ExxonMobil |
| 40 | | |
| SpectraSyn | 100 cSt C₁₀/C₁₂ Blend | ExxonMobil |
| 100 | | |

Inventive Mixed Feed PAO (MFPAO)

| | | |
|---|---|---|
| MFPAO-1 | Inventive | ExxonMobil |
| | 6 cSt Mixed Feed PAO | |
| | C₈/C₁₀/C₁₂ (10/60/30) | |
| MFPAO-2 | Inventive | ExxonMobil |
| | 6 cSt Mixed Feed PAO | |
| | C₁₀/C₁₂ (55/45) | |
| MFPAO-3 | Inventive | ExxonMobil |
| | 2 cSt Mixed Feed PAO | |
| | C₁₀/C₁₂ (55/45) | |
| MFPAO-4 | Inventive | ExxonMobil |
| | 4 cSt Mixed Feed PAO | |
| | C₁₀/C₁₂ (55/45) | |

The following Examples are directed to the similarity between mixed feed PAOs of the present invention, and those available in the art.

6 cSt PAO Comparison

| | | | | | |
|---|---|---|---|---|---|
| | | | | | Comparative |
| | Method | | | Comparative | SpectraSyn |
| Properties | (ASTM) | MFPAO-1 | MFPAO-2 | PureSyn 6 | 6LN |

Kinematic Viscosity

| | | | | | |
|---|---|---|---|---|---|
| 40° C., cSt | D 445 | 31 | 30 | 30 | 34 |
| 100° C. cSt | D 445 | 5.8 | 5.9 | 5.7 | 64 |
| Viscosity Index | D 2270 | 132 | 143 | 134 | 143 |

Appearance

| | | | | | |
|---|---|---|---|---|---|
| @ 25° C. | Visual | Clear | Clear | Clear | Clear |
| | | Colorless | Colorless | Colorless | Colorless |
| ASTM Color | D 1500 | <0.5 | <0.5 | <0.5 | <0.5 |
| APHA Color | D 5386 | 5 | 5 | 10 | 5 |
| Odor | Olfactory | None | None | None | none |

Physical Properties

| | | | | | |
|---|---|---|---|---|---|
| Specific | D 4052 | 0.827 | 0.827 | 0.827 | 0.828 |
| Gravity | | | | | |
| @ 15.6/15.6° C. | | | | | |
| Refractive | D 1218 | 1.4578 | 1.4579 | 1.4581 | 1.4580 |
| Index | | | | | |
| Molecular | GC | 528 | 533 | 527 | 562 |
| Weight (Mn) | | | | | |
| Flash Point | D 92 | 246 | 249 | 243 | 260 |
| COC ° C. | | | | | |
| Flash Point | D 93 | 230 | 239 | 224 | 243 |
| PMCC, ° C. | | | | | |
| Surface | D 971 | 28.53 | 28.91 | 28.60 | 28.72 |
| Tension | | | | | |
| dynes/cm | | | | | |
| Pour Point ° C. | D 97 Auto | −57 | −51 | −54 | −39 |
| Solubility | Calculated | 8.15 | 8.15 | 8.14 | 8.16 |
| Parameter | Fedors | | | | |
| | Correlation | | | | |
| HLB | Partition | 10 | 10 | 10 | 10 |
| Spreadability | Chaiyawat, A | 169 | 144 | 158 | 157 |
| Vitro-Skin, | et al. (GE) | | | | |
| mm²/5 mins. | | | | | |

2 and 4 cSt PAO Comparison

| | | | | | |
|---|---|---|---|---|---|
| | Method | Comparative | | Comparative | |
| Properties | (ASTM) | PureSyn 2 | MFPAO-3 | PureSyn 4 | MFPAO-4 |

Kinematic Viscosity

| | | | | | |
|---|---|---|---|---|---|
| 40° C., cSt | D 445 | 5 | 6.86 | 18 | 18.14 |
| 100° C., cSt | D 445 | 1.7 | 2.11 | 4.1 | 4.09 |
| Viscosity Index | D 2270 | na | Na | 131 | 128 |

Appearance

| | | | | | |
|---|---|---|---|---|---|
| @ 25° C. | Visual | Clear | Clear | Clear | Clear |
| | | Colorless | Colorless | Colorless | Colorless |
| ASTM Color | D 1500 | <0.5 | <0.5 | <0.5 | <0.5 |
| APHA Color | D 5386 | 10 | <5 | 10 | <5 |
| Odor | Olfactory | none | none | None | none |

Physical Properties

| | | | | | |
|---|---|---|---|---|---|
| Specific | D 4052 | 0.798 | 0.7984 | 0.82 | 0.8153 |
| Gravity | | | | | |
| @ 15.6/15.6° C. | | | | | |
| Refractive | D 1218 | 1.4418 | 1.4453 | 1.4535 | 1.4537 |
| Index | | | | | |
| Molecular | GC | 283 | 312 | 432 | 450 |
| Weight (Mn) | | | | | |
| Flash Point | D 92 | 154 | 186 | 221 | 220 |
| COC ° C. | | | | | |
| Flash Point | D 93 | 145 | 181 | 219 | 211 |
| PMCC, ° C. | | | | | |
| Surface | D 971 | 28 | 27.26 | 29.1 | 28.17 |
| Tension | | | | | |
| dynes/cm | | | | | |
| Pour Point ° C. | D 97 Auto | −66 | −57 | −63 | <−57 |
| Solubility | Calculated | 7.99 | 8.04 | 8.09 | 8.12 |
| Parameter | Fedors | | | | |
| | Correlation | | | | |
| HLB | Partition | 10 | 10 | 10 | 10 |
| Spreadability | Chaiyawat, A | 344 | 344 | 270 | 226 |
| Vitro-Skin, | et al. (GE) | | | | |
| mm²/5 mins. | | | | | |

Accordingly, the above data demonstrates the similarity in the inventive mixed feed PAOs with polydecene and polydodecene PAOs available in the art.

Sensory Testing

Skin lotion formulations were prepared utilizing comparative 6 cSt PAOs and Inventive 6 cSt Mixed Feed PAOs. A four person panel evaluated the samples on ease of emulsification, color, odor, and feel. This round of testing is inherently subjective, however all four formulations were adjudged as basically identical in all four aspects.

Skin Lotion Formulation

| Phase | Component | Wt. % |
|---|---|---|
| A | Deionized water | 62.68 |
| A | Carbopol 980 (2% solution) | 20.00 |
| A | Disodium EDTA | 0.10 |
| A | Propylene glycol | 2.00 |
| B | Promulgen D | 2.00 |
| B | 6 cSt PAO | 10.00 |
| B | Arlacel 165 | 1.50 |
| C | Triethanolamine (99%) | 0.72 |
| | To pH 6.5-7.0 | |
| D | Germaben II | 1.00 |
| | TOTAL | 100.00 |

Results

| 6 cSt PAO | MFPAO-1 | MFPAO-2 | PureSyn 6 | SpectraSyn 6 |
|---|---|---|---|---|
| Ease of | Good | Good | Good | Good |
| Emulsification | | | | |
| Color of | Good | Good | Good | Good |
| Formulation | | | | |
| Odor of | Good | Good | Good | Good |
| Formulation | | | | |
| Feel of | Good | Good | Good | Good |
| Formulation | | | | |

The skin lotion formulations were prepared by the following steps:

Step 1 Prepare phases using simple mixing.

Step 2 Heat Phases A and B each to about 70° C.

Step 3 Add Phase B to Phase A.

Step 4 Add Phase C at 25° C., cool mixture to about 50° C.

Step 5 Add Phase D and cool mixture to about 30° C., and package.

Antiperspirant Formulations

Inventive antiperspirant/deodorant (AP/DO) formulations preferably comprise aluminum or aluminum zirconium salts which serve as the antiperspirant active ingredient by plugging the apocrine glands in the underarm area. The acidic salt can cause irritation and can leave a white coating on shaved skin which is aesthetically unacceptable. The salt must also disperse and spread adequately on the skin to provide optimum AP protection.

Possible formulations comprising the inventive mixed feed PAOs include both stick and roll-on deodorant/antiperspirant formulations. Examples of possible formulations include:

POSSIBLE ROLL-ON FORMULATIONS

| | | ROLL-ON | ROLL-ON | ROLL-ON | ROLL-ON |
|---|---|---|---|---|---|
| PHASE | COMPONENT | A | B | C | D |
| A | Dow Corning | 51.29 | 31.2 | 26.2 | 16.2 |
| | 345 Fluid | | | | |
| A | Bentone Gel | 12.5 | 12.5 | 12.5 | 12.5 |
| | VS-5 PC | | | | |
| B | Mixed Feed | 10 | 30 | 35 | 45 |
| | PAO | | | | |
| C | Rezal 36GP | 20 | 20 | 20 | 20 |
| C | Talc-127PEG-8 | 2 | 2 | 2 | 2 |
| | Distearate | | | | |
| D | Dow Corning | 4 | 4 | 4 | 4 |
| | 190 Surfactant | | | | |
| D | Fragrance | 0.3 | 0.3 | 0.3 | 0.3 |
| TOTAL | | 100 | 100 | 100 | 100 |

Possible Blending Procedure:
1) Mix Phase A for about 3 min.;
2) Mix in phases B & C;
3) Mix Phase D together and add to mixture from Step 2; and

4) Mix combination from Step 3 until uniform.

POSSIBLE STICK FORMULATIONS

| PHASE | COMPONENT | STICK A | STICK B | STICK C |
|---|---|---|---|---|
| A | Mixed Feed PAO | 10 | 25 | 53.5 |
| A | Hydrogenated Caster Oil | 2.5 | 2.5 | 2.5 |
| A | PEG-8 Distearate | 1 | 1 | 1 |
| A | Stearyl Alcohol | 18 | 18 | 18 |
| B | Dow Corning 345 Fluid | 43.5 | 28.5 | 0 |
| C | Cabosil M-5 | 0.5 | 0.5 | 0.5 |
| E | Reach AZP 908 SUF | 24 | 24 | 24 |
| F | Fragrance | 0.5 | 0.5 | 0.5 |
| TOTAL | | 100 | 100 | 100 |

Possible Blending Procedure:
1) Mix and heat components of Phase A to about 85° C. or until clear;
2) Cool mixed Phase A to about 70° C.;
3) Heat Phase B to about 70° C., add to mixed Phase A (Step 2), and mix well;
4) Add Phases C and D slowly to the mixture of Step 3) while maintaining the temperature at about 70° C.;
5) Mix well until homogeneous, cool to about 56-58° C. and add Phase E;
6) Mix well and pour into stick molds or containers.

Accordingly, the inventive mixed feed PAOs are thought to provide direct replacements for neat feed PAOs within roll-on and stick formulations.

Formulations directed to the use of the inventive mixed feed PAOs in this aspect of the invention were also prepared and subjected to comparison with AP/DO formulations comprising commercially available polydecene PAOs, polydodecene PAOs, and blends of neat polydecene and polydodecene PAOs.

Solid antiperspirant compositions comprising an enhanced efficacy aluminum zirconium tetrachlorohydrexglycine active were prepared according to the following formulas wherein the inventive mixed feed PAOs were directly compared to commercially available PAOs. MFPAO-1 was utilized to produce Examples 1, 3, and 5. PureSyn 6 was utilized in Comparative Examples 2, 4, and 6.

| | | WT % | WT % | WT % |
|---|---|---|---|---|
| | | EXAMPLE | EXAMPLE | EXAMPLE |
| | | 1 & COM- | 3 & COM- | 5 & COM- |
| | | PARATIVE | PARATIVE | PARATIVE |
| | | EXAMPLE | EXAMPLE | EXAMPLE |
| PHASE | COMPONENT | 2 | 4 | 6 |
| A | PAO | 9 | 25.75 | 42 |
| A | PPG-14 | 9 | 9 | 9 |
| | Butyl Ether | | | |
| A | Hydrogenated | 2.5 | 2.5 | 2.5 |
| | Castor Oil | | | |
| A | PEG-8 | 1 | 1 | 1 |
| | Distearate | | | |
| A | Stearyl | 18 | 18 | 18 |
| | Alcohol | | | |
| B | Dow Corning | 32.5 | 16.25 | 0 |
| | 245 Fluid | | | |
| C | Cabosil M-5 | 0.5 | 0.5 | 0.5 |
| D | Talc 525 1L | 3 | 3 | 3 |
| E | Reach AZP | 24 | 24 | 24 |
| | 908 SUF | | | |
| F | Fragrance | 0.5 | 0 | 0 |
| TOTAL | | 100 | 100 | 100 |

Blending Procedure
Step 1. Mix and heat components of phase A to about 85° C. or until clear, Cool to 70° C.;
Step 2. Heat phase B to 70° C., add to phase A and mix well;
Step 3. Slowly add phases C, D, and E while maintaining 70° C.;
Step 4. Mix well until homogeneous, cool to 56-58° C. and add phase F;

Step 5. Mix well and pour into stick mold and allow to cool.

| PHASE | COMPONENT | EX. 7 | EX. 8 | EX. 9 | EX. 10 | EX. 11 |
|---|---|---|---|---|---|---|
| A | MFPAO-1 | | 20 | 10 | | |
| A | MFPAO-2 | | | | 10 | |
| A | MFPAO-3 | 20 | | | | 15 |
| A | MFPAO-4 | | 20 | | | |
| A | SpectraSyn 100 | | | | | 5 |
| A | Hydrogenated | 2.5 | 2.5 | 2.5 | 2.5 | 2.5 |
| | Castor Oil | | | | | |
| A | PEG-8 Distearate | 1.0 | 1.0 | 1.0 | 1.0 | 1.0 |
| A | Stearyl Alcohol | 18.0 | 18.0 | 18.0 | 18.0 | 18.0 |
| B | Dow Corning 345 | 33.5 | 33.5 | 43.5 | 43.5 | 33.5 |
| | Fluid | | | | | |
| C | Cabosil M-5 | 0.5 | 0.5 | 0.5 | 0.5 | 0.5 |
| D | Reach AZP 908 | 24.0 | 24.0 | 24.0 | 24.0 | 24.0 |
| | SUF | | | | | |
| E | Fragrance | 0.5 | 0.5 | 0.5 | 0.5 | 0.5 |
| TOTAL | | | | 100 | 100 | 100 |

Blending Procedure
Step 1. Mix and heat components of phase A to about 85° C. or until clear, Cool to 70° C.;
Step 2. Heat phase B to 70° C., add to phase A and mix well;
Step 3. Slowly add phases C, and D while maintaining 70° C.;
Step 4. Mix well until homogeneous, cool to 56-58° C. and add phase E;
Step 5. Mix well and pour into stick mold, cooling to about room temperature or below.
   Results

The samples were evaluated by a three person panel for emollience (feel to the skin), for the appearance of a white residue left on skin immediately after application, and for a visible white residue after a dry down time of 5 minutes after application to the skin. In each case the Examples comprising the inventive mixed feed PAOs were comparable in performance to polydecene based PAOs in the comparative formulations.

Dispersion

The wetting efficiency of the mixed feed PAOs were evaluated and compared to similar polydecene and polydodecene PAOs by methods typical in the art. In the tests conducted, the viscosity increase upon addition of the active material to the PAO was determined. The lower the dispersion viscosity increase compared to the neat viscosity, the better the wetting properties of the material for this active. Low viscosity increase is associated with good wetting, uniform spreading of the fluid, little or no air entrapment, and little or no agglomeration.

The above described REACH 908 salt was dispersed in the above inventive mixed feed PAO samples, and in the above comparative PAOs. In each case, inventive mixed feed PAOs demonstrated the same characteristics as comparative commercially available PAOs of comparable neat viscosity.

Accordingly, the inventive mixed feed PAOs demonstrate the unexpected ability to provide at least a direct replacement for commercially available PAOs.

Embodiments of the present invention include:
1a. A composition for topical application to human skin, comprising a biologically active material and a cosmetically suitable carrier, wherein the cosmetically suitable carrier comprises 5-95 wt % of a mixed feed polyolefin based on the total weight of the composition, wherein the mixed feed polyolefin comprises two or more C₆-C₂₀ copolymerized monomers, and wherein at least two of the copolymerized monomers are each present in the mixed feed polyolefin at 10 wt % or more, based on the total weight of the mixed feed polyolefin.
2a. The composition of 1a, wherein the mixed feed polyolefin consists essentially of alpha-olefin monomers.
3a. The composition of 1a or 2a, wherein the mixed feed polyolefin comprises C₈-C₁₂ copolymerized monomers.
4a. The composition of any one of any one of 1a-3a, wherein the at least two copolymerized monomers are each present in the mixed feed polyolefin at 20 wt % or more, based on the total weight of the mixed feed polyolefin.
5a. The composition of any one of 1a-4a, wherein the mixed feed polyolefin comprises at least three monomers.
6a. The composition of any one of 1a-5a, wherein the mixed feed polyolefin comprises branched monomers.
7a. The composition of any one of 1a-6a, wherein the mixed feed polyolefin consists essentially of linear alpha olefin monomers.
8a. The composition of any one of 1a-7a, wherein at least a portion of the mixed feed polyolefin has a viscosity of less than about 40 cSt at 40° C.
9a. The composition of any one of 1a-8a, wherein at least a portion of the mixed feed polyolefin has a viscosity of less than about 10 cSt at 40° C.
10a. The composition of any one of 1a-9a, wherein at least a portion of the mixed feed polyolefin has a viscosity of less than about 5 cSt at 40° C.
11a. The composition of any one of 1a-10a, wherein at least a portion of the mixed feed polyolefin has a viscosity of greater than about 100 cSt at 40° C.
12a. The composition of any one of 1a-11a, wherein at least a portion of the mixed feed polyolefin has a flash point of greater than or equal to 140° C.
13a. The composition of any one of 1a-12a, wherein at least a portion of the mixed feed polyolefin has a flash point of less than or equal to 140° C.
14a. The composition of any one of 1a-13a, wherein the mixed feed polyolefin has a refractive index of greater than or equal to 1.4.
15a. The composition of any one of 1a-14a, wherein the mixed feed polyolefin has a refractive index of greater than or equal to 1.45.
16a. The composition of any one of 1a-15a, wherein the mixed feed polyolefin has a refractive index of greater than or equal to 1.456.
17a. The composition of any one of 1a-16a, wherein the biologically active material comprises a sunscreen, an antiperspirant, a deodorant, an analgesic active, or a combination thereof.
18a. The composition of any one of 1a-17a, wherein the biologically active material comprises a zirconium-aluminum-glycine complex.
19a. The composition of any one of 1a-18a, wherein the cosmetically suitable carrier comprises an oxygen containing emollient, a liquid aliphatic hydrocarbon, a wax, a volatile silicone oil, a non-volatile silicone oil, a liquid aliphatic ester, an aliphatic alcohol, an aliphatic ether, a structurant, a gellant, polydecene, polydodence, polyoctene, or a combination thereof.
20a. The composition of any one of 1a-19a comprising a structured liquid having a firm appearance.
21a. The composition of any one of 1a-20a, wherein the composition is essentially free of volatile silicone oil.
22a. The composition of any one of 1a-21 a, wherein the composition is essentially free of cyclomethicone tetramer, cyclomethicone pentamer, or both.
23a. A method of making any of compositions 1a-22a for topical application to human skin, comprising combining a mixed feed polyolefin into a carrier to produce a cosmetically suitable carrier, wherein the cosmetically suitable carrier comprises 5-95 wt % of the mixed feed polyolefin based on the total weight of the composition,
wherein the mixed feed polyolefin comprises two or more C₆-C₂₀ copolymerized monomers, and
wherein at least two of the copolymerized monomers are each present in the mixed feed polyolefin at 10 wt % or more, based on the total weight of the mixed feed polyolefin.

All documents described herein are incorporated by reference herein, including any priority documents and/or testing procedures. As is apparent from the foregoing general description and preferred embodiments, while forms of the invention have been illustrated and described, various modifications can be made without departing from the spirit and scope of the invention. Accordingly, it is not intended that the invention be limited to the specific embodiments described in this application.

We claim:

1. A composition for topical application to human skin, comprising a biologically active material and a cosmetically suitable carrier, wherein the cosmetically suitable carrier comprises 5-95 wt % of a mixed feed polyolefin based on the total weight of the composition, wherein the mixed feed polyolefin comprises two or more C₆-C₂₀ copolymerized monomers, and wherein at least two of the copolymerized monomers are each present in the mixed feed polyolefin at 10 wt % or more, based on the total weight of the mixed feed polyolefin.

2. The composition of claim 1, wherein the mixed feed polyolefin consists essentially of alpha-olefin monomers.

3. The composition of claim 1, wherein the mixed feed polyolefin comprises C₆-C₁₄ copolymerized monomers.

4. The composition of claim 1, wherein the at least two copolymerized monomers are each present in the mixed feed polyolefin at 20 wt % or more, based on the total weight of the mixed feed polyolefin.

5. The composition of claim 1, wherein the mixed feed polyolefin comprises at least three monomers.

6. The composition of claim 1, wherein the mixed feed polyolefin comprises branched monomers.

7. The composition of claim 1, wherein the mixed feed polyolefin consists essentially of linear alpha olefin monomers.

8. The composition of claim 1, wherein at least a portion of the mixed feed polyolefin has a viscosity of less than about 40 cSt at 40° C.

9. The composition of claim 1, wherein at least a portion of the mixed feed polyolefin has a viscosity of less than about 10 cSt at 40° C.

10. The composition of claim 1, wherein at least a portion of the mixed feed polyolefin has a viscosity of less than about 5 cSt at 40° C.

11. The composition of claim 1, wherein at least a portion of the mixed feed polyolefin has a viscosity of greater than about 100 cSt at 40° C.

12. The composition of claim 1, wherein at least a portion of the mixed feed polyolefin has a flash point of greater than or equal to 140° C.

13. The composition of claim 1, wherein at least a portion of the mixed feed polyolefin has a flash point of less than or equal to 140° C.

14. The composition of claim 1, wherein the mixed feed polyolefin has a refractive index of greater than or equal to 1.4.

15. The composition of claim 1, wherein the mixed feed polyolefin has a refractive index of greater than or equal to 1.45.

16. The composition of claim 1, wherein the mixed feed polyolefin has a refractive index of greater than or equal to 1.456.

17. The composition of claim 1, wherein the biologically active material comprises a sunscreen, an antiperspirant, a deodorant, an analgesic active, or a combination thereof.

18. The composition of claim 1, wherein the biologically active material comprises a zirconium-aluminum-glycine complex.

19. The composition of claim 1, wherein the cosmetically suitable carrier comprises an oxygen containing emollient, a liquid aliphatic hydrocarbon, a wax, a volatile silicone oil, a non-volatile silicone oil, a liquid aliphatic ester, an aliphatic alcohol, an aliphatic ether, a structurant, a gellant, polydecene, polydodence, polyoctene, or a combination thereof.

20. The composition of claim 1 comprising a structured liquid having a firm appearance.

21. The composition of claim 1, wherein the composition is essentially free of volatile silicone oil.

22. The composition of claim 1, wherein the composition is essentially free of cyclomethicone tetramer, cyclomethicone pentamer, or both.

23. A method of making a composition for topical application to human skin, comprising a biologically active material and a cosmetically suitable carrier, comprising combining a mixed feed polyolefin into a carrier to produce a cosmetically suitable carrier, wherein the cosmetically suitable carrier comprises 5-95 wt % of the mixed feed polyolefin based on the total weight of the composition,
wherein the mixed feed polyolefin comprises two or more C₆-C₂₀ copolymerized monomers, and
wherein at least two of the copolymerized monomers are each present in the mixed feed polyolefin at 10 wt % or more, based on the total weight of the mixed feed polyolefin.
